(19) United States
(12) Patent Application Publication
(10) Pub. No.: US 2019/0115096 A1
(43) Pub. Date: Apr. 18, 2019

(54) PERSONAL HEALTH CARE RECORDS AGGREGATION

(71) Applicant: My Info LLC, Boca Raton, FL (US)

(72) Inventor: C.J. Wilson, Boca Raton, FL (US)

(21) Appl. No.: 15/974,575

(22) Filed: May 8, 2018

Related U.S. Application Data
(63) Continuation of application No. 14/578,375, filed on Dec. 20, 2014, now abandoned.

(51) Int. Cl. G16H 15/00; G16H 10/60; G16H 10/20

(52) U.S. Cl. CPC G16H 15/00; G16H 10/20; G16H 10/60

(57) ABSTRACT

Embodiments of the invention provide for personal health care records aggregation. In an embodiment of the invention, a method for personal health care records aggregation is provided. The method includes storing in a database, different health care records pertaining to different patients and acquiring from a pervasive device of one of the patients, an observational record pertaining to a subjective observation of the one of the patients. The method further includes date stamping the health care records and the observational record. Thereafter, an identifier of the one of the patients is received from over a computer communications network and, in response, an aggregated personal health care record of the one of the patients that includes a date sorted view of ones of the different health care records pertaining to the one of the patients and also the observational record of the one of the patients, is transmitted to a health care provider.

### BACKGROUND OF THE INVENTION

### Field of the Invention

The present invention relates to personal health care records management and more particularly to the dynamic management of personal health care records.

### Description of the Related Art

Health care records, namely medical records including a patient's medical chart, provide for the systematic documentation of the medical history of a patient and the health care afforded to the patient across a period of time with respect to health care provided by one or more health care provides. The prototypical medical record includes a variety of annotations entered over the period of time recording the observations of the health care provider or providers, the administration of drugs and therapies, recommendations for the administration of drugs and therapies, test results, diagnostics and the like. Customarily, the maintenance of complete and accurate medical record for each patient is the legal responsibility of the health care provider.

Medical records have traditionally been compiled and maintained by health care providers, but advances in online data storage have led to the development of the concept of a personal health record maintained by the patient him or herself. Several third-party providers [?] have facilitated the collection of health care data, oftentimes through the deployment of a Web site to which the end user subscribes. More recent advancements in personal health records management utilizes mobile applications resident on a pervasive device belonging to the patient, such as a smart phone or tablet computer. Data is collected on the pervasive device in that circumstance[and uploaded to centralized storage for future access

Yet, the collection of personal health care data lacks context. To wit, the general health of a patient remains heavily influenced by the environment in which the patient exists. But, the data within the personal health care record only provides data acquired when the patient is present within the health care environment or when being digitally monitored which generally differs from the ordinary environment in which the patient exists. Thus, for a health care provide unfamiliar with the activities of the patient, the personal health care record lacks context.

### BRIEF SUMMARY OF THE INVENTION

Embodiments of the present invention address deficiencies of the art in respect to personal health care records management and provide a novel and non-obvious method, system and computer program product for personal health care records aggregation. In an embodiment of the invention, a method for personal health care records aggregation is provided. The method includes storing in a database different health care records pertaining to different patients and acquiring from a pervasive device of one of the patients, an observational record pertaining to a subjective observation of the one of the patients. The method further includes date stamping the health care records and the observational record. Thereafter, an identifier of the one of the patients is received from over a computer communications network and, in response, an aggregated personal health care record of the one of the patients that includes a date sorted view of ones of the different health care records pertaining to the one of the patients and also the observational record of the one of the patients, is transmitted to a health care provider.

In one aspect of the embodiment, the identifier is disposed in a bar code, for instance a quick response (QR) code or NFC attenna. In another aspect of the embodiment, the bar code or NFC attenna is affixed to a tab attached to an article of clothing of the one of the patients. In yet another aspect of the embodiment, the observational record of the one of the patients is acquired as speech dictation provided through the pervasive device and speech recognized into text constituting the observational record. In even yet another aspect of the embodiment, the observational record is an expression by the one of the patients of how the one of the patients feels, what the patient ingested, or an activity in which the one of the patients engaged.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for personal health care records aggregation;

FIG. 2 is a schematic illustration of a health care records data processing system configured for personal health care records aggregation; and,

FIG. 3 is a flow chart illustrating a process for personal health care records aggregation.

### DETAILED DESCRIPTION OF THE INVENTION

Embodiments of the invention provide for personal health care records aggregation. In accordance with an embodiment of the invention, personal health care records entries can be accumulated in a database of personal health care records in association with a patient. Each of the health care records can pertain to treatment provided by a health care provider to the patient, diagnostic data acquired in respect to the patient by a health care provider, one or more medications or therapies prescribed and/or administered to the patient by a health care provider, one or more digital records provided by one or more monitoring device, and one or more diagnoses in respect to the health of the patient. Each of the health care records can include a date stamp indicating when the medical record had been established.

Of note, one or more additional patient subjective observational records pertaining to the state and activities of the patient outside the presence of the health care provider or digital monitoring devices can be captured in the database. The activities can include the participation in one or more different physical sports or events whether planned or unplanned, the consumption of a particular chemicals, food or beverage, or even the presence of the patient at a particular place. The state in turn can be a subjective determination by the patient of wellness such as how the patient physically or emotionally feels. Each observational record also can be time stamped. The entry of the observations can occur by way of a user interface of a mobile application in a pervasive device such as a smartphone. The user interface can include a graphical user interface, or an audio user interface into which speech dictation pertaining to an activity of the patient can be provided and converted by way of speech recognition into the requisite activity data of the additional records. The user interface and/or audio user interface can be provided remotely by a Web server, or voice gateway, or locally within the pervasive device.

Thereafter, an aggregated personal health care record can be generated by sorting the health care records and the observations in date order so as to provide context to the health care records. Optionally, the observations can be classified by parsing each observation and identifying key words associates with known classifications such as “pain observation”, “physical activity”, “meal”, “location” etc. Further, the complete health care record can be associated with a particular identifier of the patient. As such, the complete personal health care record can be accessed electronically by an inquiring health care provider or first responder by electronically matching the particular identifier to the health care record and observational records of the patient. For example, the particular identifier can be extracted from a bar code affixed to an article of clothing of the patient or disposed on a card of the patient, or embedded in the memory of a smart phone, such as within read only memory, FLASH memory, a SIM card, NFC tag or RFID tag.

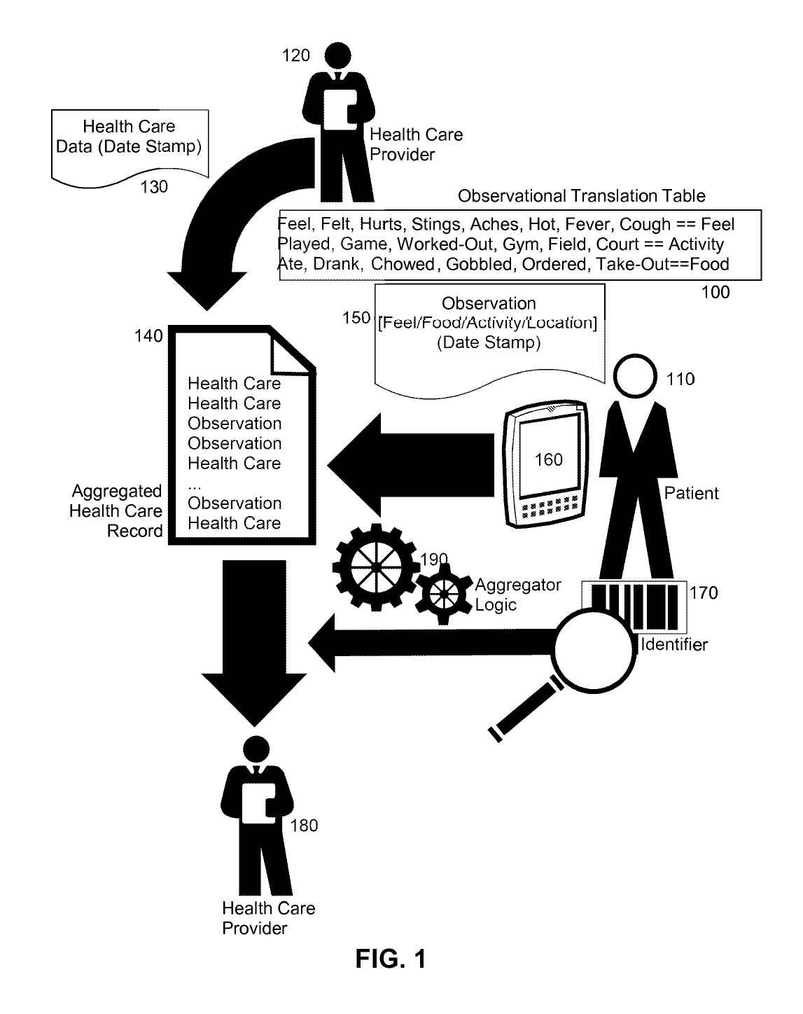

In further illustration, FIG. 1 pictorial depicts a process for personal health care records aggregation. As shown in FIG. 1, a health care provider 120 can provide health care data 130 for a patient 110 to aggregator logic 190 for aggregation into an aggregated personal health care record 140. The patient 110 also can provide one or more observations 150 to the aggregator logic 190 for inclusion into the aggregated health care record 140. In particular, the patient 110 can provide the observations 150 by way of a pervasive device 160 such as a smart phone either through text entry or voice entry.

The health care data 130 can include data pertaining to the health care of the patient 110. The observations 150 in contrast can include data pertaining to the subjective observations of the patient 110 regarding the state of the patient 110, for example how the patient feels, the activities undertaken by the patient 110, foods and beverages consumed by the patient 110, and the like. To wit, the observations 150 can be classified in accordance with keywords present in the observations 150 mapped to subjective observational classes in a translation table 100. Both the health care data 130 and the observations 150 can be date stamped or date and time stamped to indicate a time or date at which the health care data 130 or the observations 150 are collected.

Thereafter, an identifier 170 associated with the patient 110 can be received by the aggregator logic 190. For example, the identifier 170 can be a bar code or RFID tag affixed to an article of clothing of the patient 110 or to a personal article of the patient 110 including the pervasive device 160. Based upon the identifier 170, the aggregator logic 190 can identify the patient 110 so as to retrieve the aggregated health care record 140 for the patient 110. In particular, the aggregated health care record 140 can include a sorted listing of the health care data 130 and observations 150. Optionally, the inclusion of the observations 150 can be filtered according to class of observation. Specifically, the health care data 130 and observations 150 can be sorted according to the date stamp associated with each of the health care data 130 and the observations 150. Finally, the aggregator logic 190 can provide the aggregated health care record 140 to a health care provider [or anyone] 180 so as to provide a contextual record of the patient 110 such that the observations 150 provide context for the health care data 130.

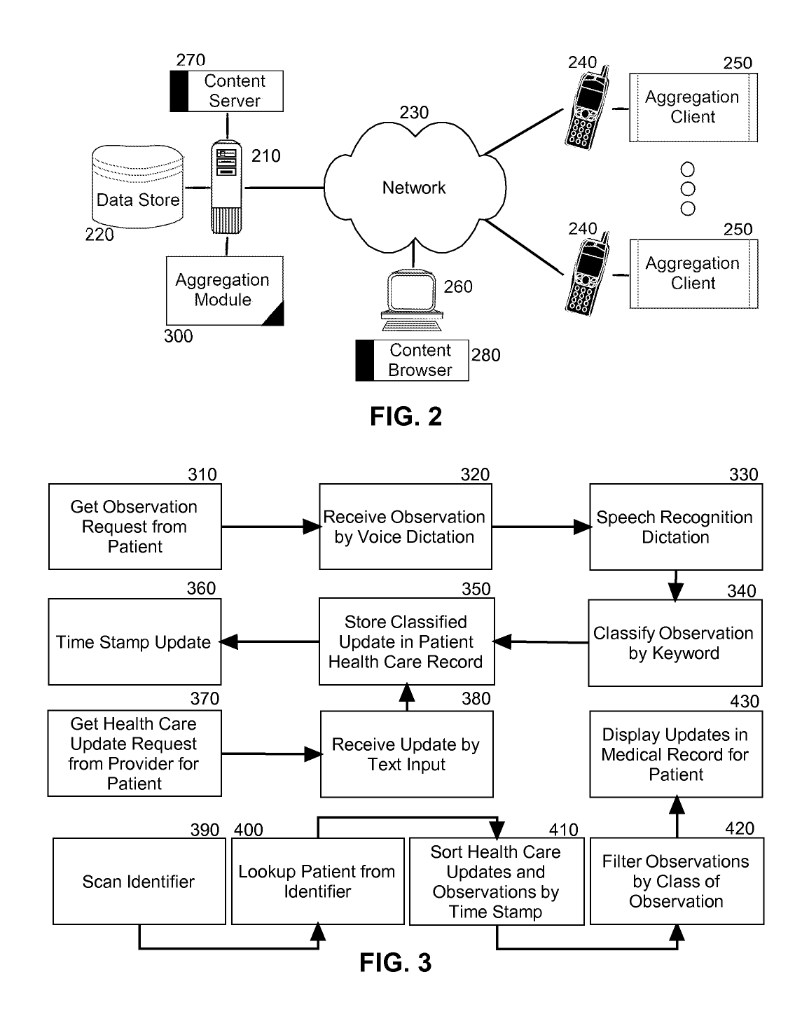

The process described in connection with FIG. 1 can be implemented within a health care records data processing system. In yet further illustration, FIG. 2 schematically shows a health care records data processing system configured for personal health care records aggregation. The system can include a host computing system 210 that can include one or more computers, each with memory and at least one processor. The host computing system 210 can be communicatively coupled to different pervasive devices 240, for instance different smart phones, over a computer communications network 230.

The host computing system 210 can support the operation of an aggregation module 300. The aggregation module 300 can include program code enabled upon execution in the memory of the host computing system 210 to record health care data in a data store 220 in association with different patients. The program code of the aggregation module 300 additionally can be enabled upon execution in the memory of the host computing system 210 to record observations in the data store 220 in association with the different patients. The observations particularly can be received through an aggregation client 250 executing in respective ones of the pervasive devices 240. In this regard, the observations can be voice dictated and speech recognized, either locally in a receiving one of the pervasive devices 240, remotely in a voice server, or provided textually [or photos] through the aggregation clients 250.

Of note, the program code of the aggregation module 280 is enabled upon execution in the memory of the host computing system 210 to receive an identifier for one of the patients through a user interface provided by a content server 270 to a content browser 280 executing in a client computer 260. The identifier can be used to identify a particular patient and to retrieve from the data store 220 an aggregated health care record of the particular patient. The aggregated health care record can include both health care data and one or more observations for the particular patient sorted according to date and/or date and time stamp for each of the health care and observations. In this way the aggregated health care record can provide a context for the included health care data.

In even yet further illustration of the operation of the aggregation module 300, FIG. 3 is a flow chart illustrating a process for personal health care records aggregation. Beginning in block 310, an observation update request can be received from a patient and in response, an observation can be received by way of voice dictation in block 320. In block 330, the voice dictation can be speech recognized into textual data in block 340 the textual data can be parsed to identify keywords indicative of a particular class of observation. Thereafter, in block 350 the textual data can be stored in a health care record for the patient. Finally, in block 360 the activity update can be date stamped and optionally, time stamped.

In addition to the observation update, in block 370 a health care update request can be received from a health care provider of the patient. In block 380 in response to the request, a textually input health care update can be received and in block 350 the textual data can be stored in a health care record for the patient. Finally, in block 360 the health care update can be date stamped and optionally, time stamped.

Of note, in block 390, an identifier can be scanned and in block 400, a patient can be determined from the identifier. In block 410 the health care data and observations for the determined patient can be sorted according to date stamp and optionally in block 420 the observations can be filtered according to specified observation. Finally, in block 430, the sorted health care data and optionally filtered observations can be presented in a user interface as an aggregated health care record for the determined patient.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

I claim:

1. A method for personal health care records aggregation, the method comprising:
storing in a database, different health care records pertaining to different patients;
acquiring from a pervasive device of one of the patients, an observational record pertaining to a subjective observation of the one of the patients;
date stamping the health records and the observational record;
receiving an identifier of the one of the patients from over a computer communications network; and
transmitting to a health care provider in response to the receipt of the identifier, an aggregated personal health care record of the one of the patients including a date sorted view of ones of the different health care records pertaining to the one of the patients and also the observational record of the one of the patients.

2. The method of claim 1, wherein the identifier is disposed in a bar code.

3. The method of claim 1, wherein the bar code is affixed to a tab attached to an article of clothing of the one of the patients.

4. The method of claim 1, wherein the observational record is an expression by the one of the patients of how the one of the patient feels.

5. The method of claim 1, wherein the observational record is acquired as speech presented through the pervasive device and speech recognized into text constituting the observational record.

6. The method of claim 1, wherein the observational record is an expression by the one of the patients of a food eaten by the one of the patients.

7. The method of claim 1, wherein the observational record is an expression by the one of the patients of an activity performed by the one of the patients.

8. A health care records data processing system configured for personal health care records aggregation, the system comprising:
a host computing system comprising one or more computers each with memory and at least one processor;
a data store coupled to the host computing system; and,
an aggregation module executing in the memory of the host computing system, the module comprising program code enabled upon execution in the memory of the host computing system to store in the data store, different health care records pertaining to different patients, to acquire from a pervasive device of one of the patients from over a computer communications network coupled to the host computing system, an observational record pertaining to a subjective observation of the one of the patients, to date stamp the health care records and the observational record, to receive from over the computer communications network an identifier of the one of the patients, and to transmit to a health care provider in response to the receipt of the identifier, an aggregated personal health care record of the one of the patients including a date sorted view of ones of the different health care records pertaining to the one of the patients and also the observational record of the one of the patients.

9. The system of claim 8, wherein the identifier is disposed in a bar code.

10. The system of claim 8, wherein the bar code is affixed to a tab attached to an article of clothing of the one of the patients.

11. The system of claim 8, wherein the observational record is an expression by the one of the patients of how the one of the patient feels.

12. The system of claim 8, wherein the observational record is an expression by the one of the patients of a food eaten by the one of the patients.

13. The system of claim 8, wherein the observational record is an expression by the one of the patients of an activity performed by the one of the patients.

14. The system of claim 7, wherein the information pertaining to the activity of the one of the patients is acquired by location determination logic disposed within the pervasive device.

15. A computer program product for personal health care records aggregation, the computer program product comprising a computer readable storage medium having program instructions embodied therewith, the program instructions executable by a device to cause the device to perform a method comprising:
storing in a database, different health care records pertaining to different patients;
acquiring from a pervasive device of one of the patients, an observational record pertaining to a subjective observation of the one of the patients;
date stamping the health care records and the observational record;
receiving an identifier of the one of the patients from over a computer communications network; and
transmitting to a health care provider in response to the receipt of the identifier, an aggregated personal health care record of the one of the patients including a date sorted view of ones of the different health care records pertaining to the one of the patients and also the observational record of the one of the patients.

16. The computer program product of claim 15, wherein the identifier is disposed in a bar code.

17. The computer program product of claim 15, wherein the bar code is affixed to a tab attached to an article of clothing of the one of the patients.

18. The computer program product of claim 15, wherein the observational record is an expression by the one of the patients of how the one of the patient feels.

19. The computer program product of claim 15, wherein the observational record of the one of the patients is acquired as speech dictation presented through the pervasive device and speech recognized into text constituting the observational record.

20. The computer program product of claim 15, wherein the observational record is an expression by the one of the patients of food eaten by the one of the patients.
